(19) United States
(12) Patent Application Publication
(10) Pub. No.: US 2005/0113816 A1
(43) Pub. Date: May 26, 2005

(54) Instrument guide with capture and release in an image plane

(76) Inventors: Willet F. Whitmore III, Longboat Key, FL (US); Craig Cermak, Riverside, IA (US)

Correspondence Address: Merchant & Gould P.C., P.O. Box 2903, Minneapolis, MN, 55402-0903, US

(21) Appl. No.: 10/851,030

(22) Filed: May 21, 2004

Related U.S. Application Data
(60) Provisional application No. 60/472,749, filed on May 23, 2003.

(51) Int. Cl. A61B 18/00

(52) U.S. Cl. 606/23; 606/28; 606/130; 606/41

(57) ABSTRACT

An instrument guide with capture and release in an imaging plane is disclosed. The instrument guide includes a securing portion, an instrument engaging portion, and a rotatably adjustable portion of the instrument engaging portion allowing rotational adjustment of the guide bath with respect to the securing position.

Ultrasound is an established imaging tool for the diagnosis and treatment of human diseases. In addition to pure imaging, ultrasound is used for real time needle guidance that enables accurate targeting of internal structures during interventional procedures such as biopsy, drainage or focal therapies that can be delivered through a needle or small diameter probe. There are two fundamental techniques for ultrasound guided needle placement. The first is the “freehand” method where the ultrasound imaging transducer and needle are separate and independently movable throughout the procedure. In this method the needle and path to the target organ are visualized by a skilled operator who holds the transducer in one hand while advancing the needle with the other. The coordination and visual and spatial orientation skills required to simultaneously maintain the image plane in line with the needle, keep the target in view, maintain orientation to surrounding structures and manipulate the needle are substantial and demanding. It is not a technique that suits everyone and there is a steep and prolonged learning curve. Its major advantage as a technique is the complete versatility allowed by total freedom of movement of both the needle and the ultrasound imaging transducer. The second method is to use a needle guide that has a known orientation to the ultrasound imaging transducer. Such guides may be built into the transducer or come as an accessory that is attached if desired. They have been made as either reusable or single use sterile disposable products. These guides are designed to keep any needle passing through them on a known path in the image plane* *and thus eliminate many of the difficult variables inherent in the freehand technique. Typically, software is supplied by the ultrasound manufacturer that shows a virtual guide path and depth index on the real time image. This method is less reliant on user intuition, skills and experience and has a shorter learning curve, but has several limitations that have hampered universal acceptance:
1) The freehand technique allows the operator to choose any size needle without restriction whereas the needle guide requires a close tolerance path for each size needle to work properly. For this reason the makers of needle guides have devised various methods that typically require significant manipulation of the guide or multiple parts to permit a range of needle sizes to be accommodated within their guides.
2) The freehand technique allows the operator complete freedom of choice for entry site of the needle and angle of attack to the target whereas the needle guides in general are very restricting typically offering only a single angle of attack in relation to the ultrasound transducer. To address this restriction some re-usable needle guides have been designed to offer up to three different angles of attack that again require significant manual manipulation and are considered cumbersome by some users.
   **Pat. No. 6,203,499 is an exception by virtue of a slot that is aligned with the image plane that permits infinite angular adjustment within a 45 degree range. In this case the angle of attack in the image plane is not fixed to a known previously determined path or paths. Nor is the needle fully engaged in the guide but merely free floating in the open sided slot. The slot is not adjustable in width and therefore is only useful for a very limited range of needle sizes.
3) The freehand technique allows the operator complete freedom with the needle once it is in the desired location. Whereas if a guide is used the needle must be detached to continue the procedure and this inevitably has required additional sometimes awkward and time consuming steps.
4) The needle guide technique introduces new problems unrelated to the freehand method. These problems include: the requirement for a secure attachment to the transducer that does not interfere with transducer function; the possible requirement for added needle length (to allow for the length of the guide); the issue of “dead space” that can exist between the exit of the needle tip from the guide and the entry into the skin and allow misdirection of the needle; and the added cost to the user. The added expense may be balanced by the need for a less experienced operator and fewer complications for the patient, but all are relevant design issues.

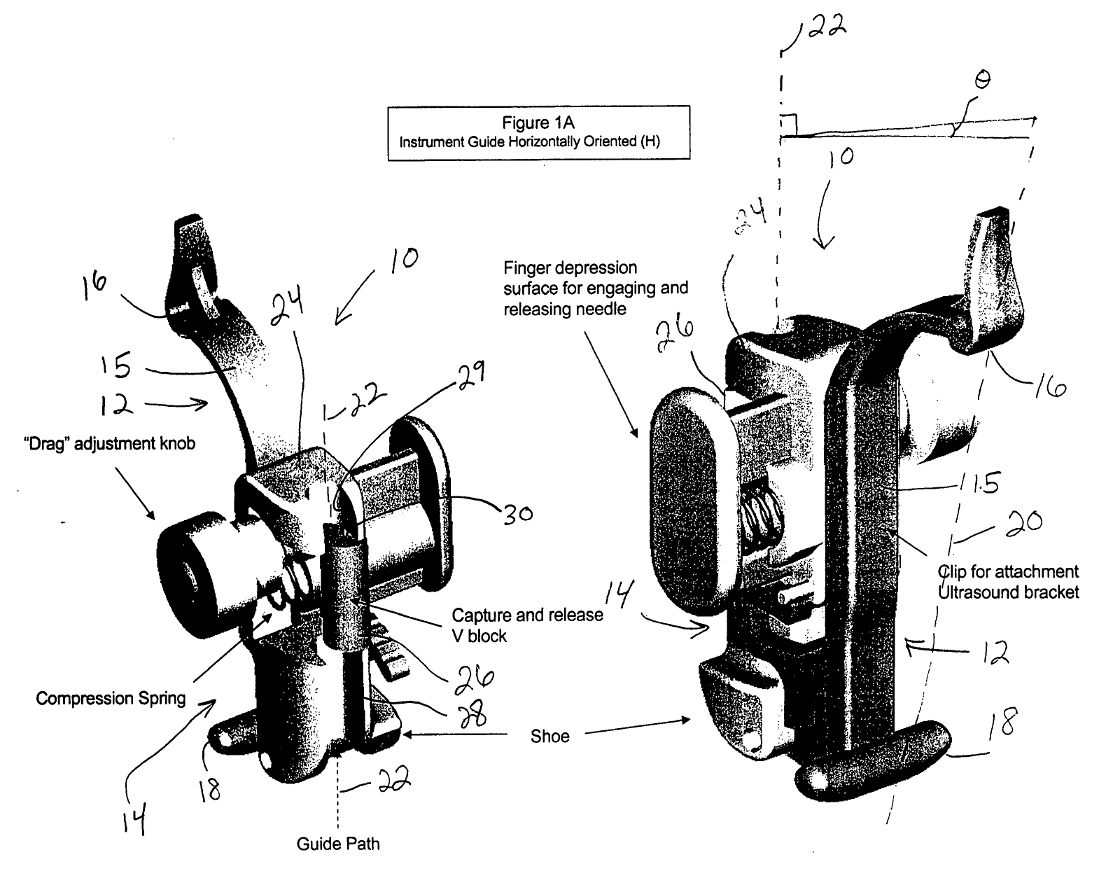

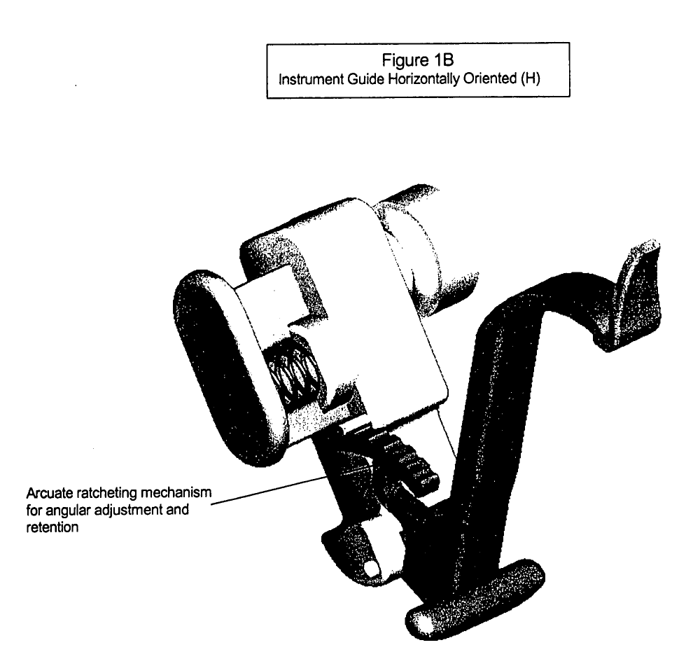

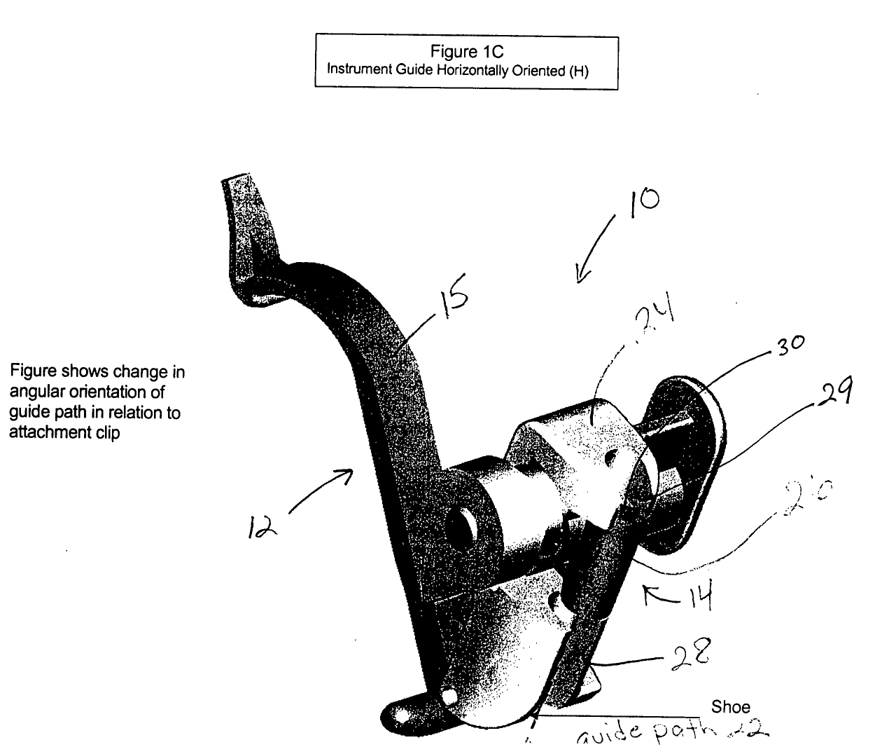

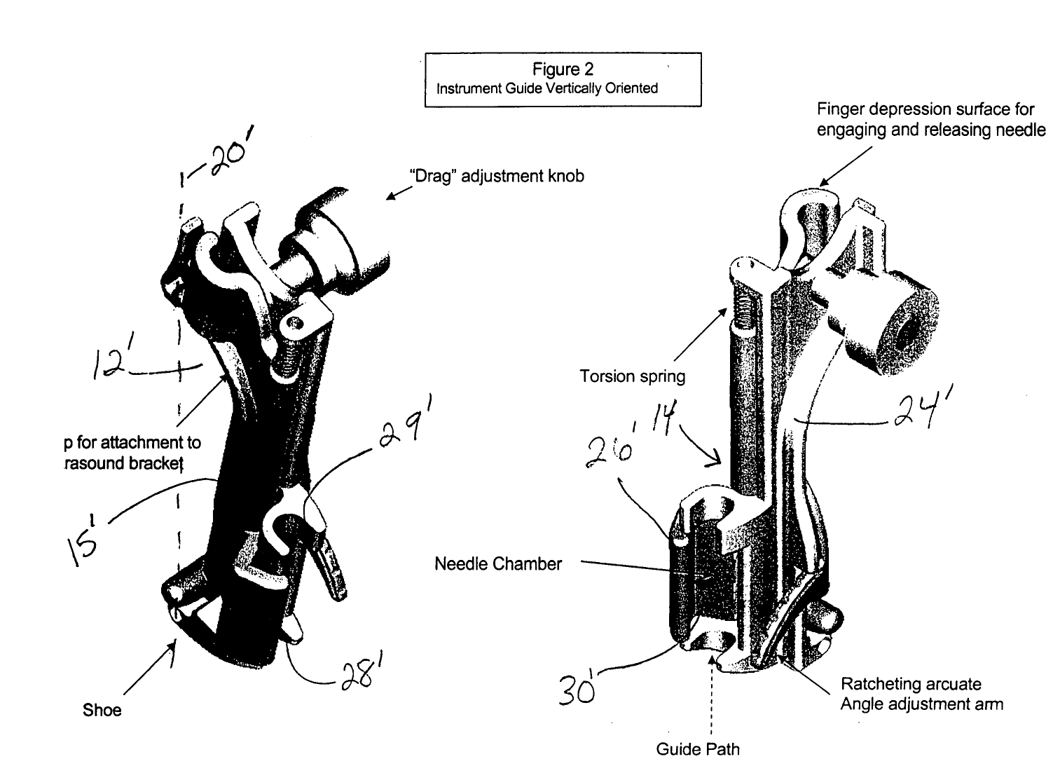

The present invention relates to a new and original design for a needle guide that efficiently marries the advantages of the freehand technique with the inherently greater control and security of the needle guide technique while addressing the design issues noted above. This guide is open to accept any needle, probe or catheter (i.e. instruments) with diameters including but not limited to all of the common sizes of 27 gauge up to 8.5 French with a vernier type user adjustable “drag” on the instrument. It uses no additional parts and requires only a single push with a finger to very quickly engage or disengage the instrument while maintaining view of the instrument in the image plane. The angle of attack for the instrument in the image plane can be user selected over an angular range by virtue of an arcuate ratcheting mechanism that also can maintain the angle chosen. The “dead space” problem is eliminated by virtue of a curved shoe at the base of the guide. The guide is short in comparison to currently used competing guides and should not compromise needle length. It can be made of single use injection molded parts and by virtue of simplicity and low cost components should be cost competitive. Two different embodiments of this design are shown in the attached FIGS. 1A, 1B, 1C, and 2.

The present invention relates to a needle guide that can be operatively secured to an ultrasound transducer that functions in an in plane relationship to an imaging plane and that has a single cavity for engaging multiple different needle diameters without the requirement for additional parts or attachments. This guide may have a reciprocating mechanism that can securely engage and accurately guide and quickly release a needle, probe or catheter in an imaging plane. The angle of approach in the chosen image plane may be adjusted by the operator before or during a procedure and may be fixed as desired The frictional resistance of the needle to sliding through the guide may be adjustable by the operator. The guide may be made disposable or reusable of a variety of materials for manufacture that may be chosen accordingly.

Referring to FIGS. 1A-1C, a needle guide 10 may include a securing portion 12 and an instrument engaging portion 14. Securing portion 12 is preferably configured to releasably secure needle guide 10 to an imaging transducer. Securing portion 12 may comprise an open clip 15 having first and second ends 16,18. An angle θ between a line 20 connecting first and second ends 16,18 and an instrument guide path 22 of instrument engaging portion 14 is preferably less than 45°, for example less than 20°. In one embodiment, line 20 and guide path 22 are aligned with one another. In one embodiment, line 20 and guide path 22 are contained within a single plane.

Instrument engaging portion 14 is preferably configured to operatively secure an instrument, such as a biopsy needle, along guide path 22. Engaging portion 14 may comprise first and second engaging portions 24,26. First engaging portion 24 may include at least one surface 28 preferably shaped to accommodate a portion of an instrument. First engaging portion 24 may include a second surface 29 spaced apart along the guide path 22 from surface 28 and preferably shaped to accommodate a portion of the instrument. Second engaging portion 26 preferably includes a surface 30 also preferably shaped to accommodate a portion of the instrument. First and second engaging portions 24,26 may cooperate to operatively secure the instrument. For example, an instrument may be compressed between (a) surface 30 and (b) surface 28 and, optionally, surface 29. In one embodiment, surfaces 28-30 contact the instrument in a three-point contact configuration. Preferably, the first and second portions 24,26 are configured to receive an instrument laterally with respect to the guide path 22.

Engaging portion 14 may be configured to allow motion, for example rotation, of guide path 22. Preferably, guide path 22 may be rotated with respect to securing portion 22. In one embodiment, a rotational motion of guide path 22 is accompanied by a rotational motion of at least one of first and second engaging portions 24,26 so that an orientation of guide path 22 relative to the first and second engaging portions 24,26 does not change as guide path 22 is rotated. An example of this may be seen upon comparing FIGS. 1A and 1C.

Referring to FIG. 2, a second embodiment of an instrument guide may include a securing portion 12′ and an instrument engaging portion 14′. Labeled elements of FIG. 2 having similar reference numerals with elements of FIGS. 1A-1C may have similar functions with those elements.

1. An instrument guide, comprising:
a securing portion for securing the instrument guide to an imaging transducer;
an instrument engaging portion for operatively securing an instrument to the instrument guide along a guide path;
wherein, at least a first portion of the instrument engaging portion is rotatably adjustable with respect to the securing portion to allow corresponding rotational adjustment of the guide path with respect to the securing portion.

2. The instrument guide of claim 1, wherein the instrument is a biopsy needle.

3. The instrument guide of claim 1, wherein the instrument is configured to aspirate a material.

4. The instrument guide of claim 1, wherein the instrument is configured to heat or cool a tissue.

5. The instrument guide of claim 4, wherein the instrument is configured to emit microwaves.

6. The instrument guide of claim 4, wherein the instrument is a cyroprobe.

7. The instrument guide of claim 1, wherein the instrument is configured to introduce a substance to a tissue.

8. The instrument guide of claim 7, wherein the substance comprises ethanol.

9. The instrument guide of claim 1, wherein the instrument is configured to emit radio waves.
